SEQUENCE LISTING (4 sequences)

SEQ ID NO: 1 - DNA, 20 bp, Homo sapiens
tcctggagaggaagccaagt

SEQ ID NO: 2 - DNA, 20 bp, Homo sapiens
aggacctctccttcggttca

SEQ ID NO: 3 - DNA, 600 bp, Homo sapiens
  misc_feature (502)..(503): n=unknown variation
tatagagcagatatacagcctattaagcgtcgtcactaaaacataaaacatgtcagcctttcttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaanntttgtggggggtacgaaaaggcggaaagaaacagtcatttcgtcacatgggcttggttttcagtcttataaaaaggaaggttctctcggttagcgac

SEQ ID NO: 4 - DNA, 600 bp, Homo sapiens
  misc_feature (502)..(503): n=unknown variation
atatctcgtctatatgtcggataattcgcagcagtgattttgtattttgtacagtcggaaagaattggaatgagcggggtcagacagggctgcactgaaggagctgggagatttctgcatgtctggtctgtgccgccgccgccgccctctcccctaagggacgcgggggcctggagtcccggcgagtctaaggacctctccttcggttcacaggaagacgggagggggccatagggtaggttccgctagtcaggtcttgaccgagagccttcgcgagcccgtttctgacgcttcttcttttctgtagaccgcctttggacacgcggaccccgccaccttgagcccctcctctccctccctagtctgtcctctcacccctgatgggggagacgagggtttaaccccgtcgaaggacccaaaggctaaaagagtaaaggcacccattttttgggacgggggtggcccgaatgcgttaaaaaaattcccctctcctccctttttnnaaacaccccccatgcttttccgcctttctttgtcagtaaagcagtgtacccgaaccaaaagtcagaatatttttccttccaagagagccaatcgctg

(19) United States
(12) Patent Application Publication
(10) Pub. No.: US 2003/0119891 A1
(43) Pub. Date: Jun. 26, 2003

(54) Polyamide modulators of COX2 transcription

(76) Inventors: Dennis P. Phillion, St. Charles, MO (US); Barry L. Burnette, High Ridge, MO (US); Barbara A. Schweitzer, Webster Groves, MO (US); Kathleen S. Crowley, Webster Groves, MO (US); James K. Bashkin, St. Louis, MO (US); Scott S. Woodard, Manchester, MO (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 10/289,794

(22) Filed: Nov. 7, 2002

Related U.S. Application Data
(60) Provisional application No. 60/419,405, filed on Oct. 18, 2002.
(60) Provisional application No. 60/344,859, filed on Nov. 7, 2001.

(51) Int. Cl. A61K 31/4178; C12N 5/02

(52) U.S. Cl. 514/397; 435/375

(57) ABSTRACT

Processes and compositions are provided for regulating COX2 gene expression in a cell. The process includes selecting a polyamide composition comprising N-methyl pyrrole (Py) and N-methyl imidazole (Im) to provide specific binding to DNA at a COX2 gene target site in a cell. The polyamide is then combined with the cell containing the COX2 gene, wherein the polyamide binds to the COX2 gene promoter target site and regulates transcription of the COX2 gene.

### CROSS-REFERENCE TO RELATED APPLICATION

This application claims priority from U.S. application Serial No. 60/419,405, filed Oct. 18, 2002 and U.S. application Serial No. 60/344,859, filed Nov. 7, 2001.

### FIELD OF THE INVENTION

The present invention relates to the regulation of COX2 gene expression. More particularly, the enhancement or reduction in the transcription of the COX2 gene using polyamides designed to bind to transcription factor binding sites in the promoter region of the COX2 gene.

### BACKGROUND OF THE INVENTION

An understanding of the synthesis, the analysis, and the manipulation of DNA has led to an explosion of opportunities for the diagnosis and treatment of various illnesses and conditions. The specific interaction of proteins, such as transcription factors, with DNA controls the regulation of genes, and hence, the regulation of cellular processes as well. Roeder, R. G., TIBBS. 9, 327-335 (1996). A wide variety of human conditions ranging from cancer to viral infection arise from malfunctions in the biochemical machinery that regulates gene expression. (R. Tjian, Sci. Am., 2, 54-61 (1995).) Therefore, researchers have focused on identifying specific sequences of DNA that, when expressed, as a result of biochemical malfunction or otherwise, cause disease, defect, and discomfort. This research has led to a better understanding of particular genetic processes, and the ways to treat and deal with theses processes when they run awry.

In recent years, researchers have learned that certain chemical compounds can be used to regulate the phenotypic effects of the genetic machinery. The expression of proteins, the end product of nucleic acid translation, can be controlled by the application of certain natural and synthetic compounds. The discovery and application of these chemicals has been to the benefit of both research and therapeutics. In research, these molecules can be used to modulate the activity of a particular gene in order to identify the function and cellular characteristics of that particular gene. In therapeutics, these molecules can be used to inhibit the proliferation of cells which may act as pathogens, where proliferation has an adverse effect on the host, or to combat life threatening diseases which result from misregulation in transcription.

It is well known that chemical compounds known as polyamides can be used to control gene expression due to their high affinity for DNA. Polyamides comprise polymers of amino acids covalently linked by amide bonds. Specific polyamides that target unique DNA sequences can be used to suppress or enhance the expression of particular genes, while not affecting the expression of others.

It has become known that certain oligomers of nitrogen heterocycles can be used to bind to particular regions of double stranded DNA. Particularly, N-methyl imidazole (IM) and N-methyl pyrrole (Py) have a specific affinity for particular bases. This specificity can be modified based upon the order in which these two compounds are linked. It has been shown that there is specificity in that G/C is complemented by Im/Py, C/G is complemented by Py/Im, and A/T and T/A are redundantly complemented by Py/Py. In effect, N-methyl imidazole tends to be associated with guanosine, while N-methyl pyrrole is associated with cytosine, adenine, and thymidine. By providing for two chains of the heterocycles, as 1 or 2 molecules, a 2:1 complex with double stranded DNA is formed, with the two chains of the oligomer antiparallel, where G/C pairs have Im/Py in juxtaposition, C/G pairs have Py/Im, and T/A pairs have Py/Py in juxtaposition. The heterocycle oligomers are joined by amide (carbamyl) groups, where the NH may participate in hydrogen bonding with nitrogen unpaired electrons, particularly of adenine.

Polyamides may be synthesized to form hair-pin compounds by incorporating a compound, such as gamma-aminobutyric acid, to allow a single polyamide to form a complex with DNA. Such a structure has been found to significantly increase the binding affinity of the polyamide to a target sequence of DNA.

More recently it has been discovered that the inclusion of a new aromatic amino acid, 3-hydroxy-N-methylpyrrole (Hp), when incorporated into a polyamide and paired opposite Py, provides the means to discriminate A-T from T-A. White S., et al., Nature 391 436-438 (1998). Unexpectedly, the replacement of a single hydrogen atom on the pyrrole with a hydroxy group in an Hp/Py pair regulates the affinity and the specificity of a polyamide by an order of magnitude. Utilizing Hp together with Py and Im in polyamides to form four aromatic amino acid pairs (Im/Py, Py/Im, Hp/Py, and Py/Hp) provides a code to distinguish all four Watson-Crick base pairs in the minor groove of DNA.

Expression of a gene occurs when transcription compounds such as activators, transcription binding proteins, transcription factors, and the like bind to specific locations in the gene's promoter region known as transcription binding sites and either initiate or inhibit the process of DNA transcription. If polyamides were designed to bind to specific transcription binding sites in a gene's promoter region, the administration of such polyamides may prevent the transcription compounds of a cell from binding to the transcription binding sites, thereby resulting in modulation of a gene expression.

### SUMMARY OF THE INVENTION

Among the various aspects of the present invention, therefore, is the provision of a process to regulate the expression of a COX2 gene using a polyamide compound, the provision of a process to enhance the expression of a COX2 gene using a polyamide compound, the provision of a process to suppress the expression of a COX2 gene using a polyamide compound, and the invention is the provision of polyamide compounds that bind to transcription binding sites in the COX2 gene promoter region.

Briefly, therefore, the present invention is directed to a process for regulating COX2 gene expression in a cell. The process comprises selecting a polyamide comprising N-methyl pyrrole (Py) and N-methyl imidazole (IM) to provide specific binding to DNA at a COX2 gene promoter target site in the cell and combining the polyamide and the cell containing the COX2 gene. The polyamide then binds to the COX2 gene promoter target site and regulates transcription of the COX2 gene.

The present invention is further directed to a polyamide compound for regulating COX2 gene expression. The polyamide comprises N-methyl pyrrole (Py) and N-methyl imidazole (IM) and specifically binds to a COX2 gene promoter region of DNA.

Other objects and features of this invention will be in part apparent and in part pointed out hereinafter.

### BRIEF DESCRIPTION OF THE DRAWINGS

FIG. 1a is an illustration of a human Ets-1 transcription factor bound to the major groove of a DNA helix.

FIG. 1b is an illustration of the Ets-1 binding site sequence in the COX2 promoter region and the binding sites of polyamides of the present invention.

FIG. 2 is a schematic of the COX2 promoter sequence identifying the transcription factor binding locations and the binding sites of polyamides of the present invention.

FIG. 3 is a bar graph illustrating the effect of arachidonic acid on the expression of PGE₂ in the presence of polyamides. Added arachidonic acid (aa) had no effect on relative expression of PGE₂ in the presence of polyamides. Mixture 1 dramatically enhanced PGE₂ levels [(−)aa, unpaired t-test P=0.0001]. Mixture 2 inhibited PGE₂ levels by 41% [(−)aa, unpaired t-test P=0.01]. N=3 for mixtures 1 & 2, n=4 for (+)IL-1b.

FIG. 4 is a bar graph illustrating the deconvolution of Mixture 1 to illustrate the effect of different polyamide combinations that result in enhanced PGE₂ levels. Mixture 1 was deconvoluted to determine which polyamide combinations led to enhanced PGE₂ levels. Combinations with the LEF1 polyamide PA3 enhanced PGE₂ levels. Compound key: PA1 (Ets-1, Im-Im-Py-Py-γ-Py-Im-Py-Py-β-Dp); PA2 (TATA Box, Im-Py-Py-Py-Im-γ-Py-Py-Im-Py-Py-β-Dp); PA3 (LEF1, Im-Py-Py-β-Im-Py-Im-γ-Py-Im-Py-β-Im-Py-Py-β-Dp); PA4 (LEF-1, Im-Py-Py-Py-Im-γ-Py-Im-Im-Im-Py-β-Dp); PA5 (Ets-1, Im-Im-Py-Im-γ-Py-Py-β-Py-β-Dp); PA6 (CRE, Im-Py-Py-Im-γ-Py-Im-Py-Py-β-Dp). Mixture 1 PA1, PA2, PA3, PA4. Mixture 2=PA1, PA2, PA5, PA6. SS1=Mixture 1; SS2=PA1, PA2, PA3; SS3=PA1, PA2, PA4; SS4=PA1, PA3, PA4; SS5=PA2, PA3, PA4; SS6=PA3, PA4; SS7=PA1, PA2.

FIG. 5 is a bar graph illustrating the enhancement and suppression of COX2 protein levels resulting from the administration of polyamides. COX2 Protein levels were enhanced 700% by Mixture 1 (unpaired t-test P=0.0009) and inhibited 35% by Mixture 2 (unpaired t-test P=0.06). Mixture 2 provided similar levels of inhibition of COX2 protein and PGE₂. N=3 for Mixtures 1 & 2, n=4 for (+)IL-1, n=2 for (−)IL-1β.

FIG. 6a is a bar graph illustrating the Northern Blot analysis of COX2 mRNA levels resulting from the administration of polyamides. Northern Blot Analysis of COX2 mRNA levels showed enhancement by mixture 1 and inhibition by mixture 2. These results were in agreement with protein and PGE₂ levels.

FIG. 6b is a photograph of a Northern Blot analysis of COX2 mRNA.

FIG. 7 is a bar graph illustrating the effect of polyamides on ICAM1 levels. The polyamides are selective for COX2: Mixture 1 had minimal effect on ICAM1 level, and Mixture 2 had no effect

FIG. 8 is a bar graph illustrating the effect of polyamides on IL-6 levels. The polyamides are somewhat selective, as Mixture 1 increased IL-6 production but much less than for COX2. Mixture 2 had no effect.

### DETAILED DESCRIPTION OF THE PREFERRED EMBODIMENTS

In accordance with the present invention, it has surprisingly been discovered that polyamides may be designed, synthesized, and utilized to regulate the transcription of the COX2 gene. More particularly, the present invention provides a process for enhancing or suppressing the transcription of the COX2 gene by utilizing polyamides that bind to transcription factor binding sites present in the COX2 promoter sequence. The present invention thereby provides a novel process to enhance or suppress the production of COX2 protein and PGE₂.

The present invention relates to the combination and use of polyamides and similar chemical compounds to enhance or inhibit the expression of the COX2 gene. Polyamides with a particular binding specificity were designed to bind to DNA minor groove regions in order to disrupt the binding of transcription factors that are known to bind specific sequences in the human COX2 promoter. The demonstrated result is the ability to manipulate COX2 gene expression through the direct control of the transcription of COX2 mRNA, thereby affecting the quantity of translated COX2 protein as well as the production of prostaglandin E₂ (PGE₂).

In general, polyamides are designed and synthesized to selectively bind at five transcription binding factors located in the promoter region of the COX2 gene. Research studies, outlined in the examples below, were conducted and the enhancing or inhibitory characteristics of the tested polyamides were determined. The COX2 transcription factor binding sites studied include Ets-1, CRE, TATA box, NFkB, and LEF-1 binding sites. By utilizing polyamides designed to target specific binding sites the transcription of the COX2 gene may be selectively enhanced or suppressed.

The research described below determined that cells treated with polyamides that targeted the Ets-1, CRE, and TATA box binding sites suppressed COX2 mRNA levels and production of PGE₂ and COX2 protein. When cells were treated with polyamides that targeted the NFkB and LEF-1 binding sites, however, the COX2 mRNA levels and production of PGE₂ and COX2 protein were either unaffected or significantly increased.

Polyamides were evaluated as inhibitors of COX2 transcription in interleukin-1β (IL-1β) stimulated human synovial fibroblasts, with some related work carried out in differentiated U937 cells. The purpose of this work was to determine how well polyamides could inhibit the transcription of a targeted gene in a cellular system, and whether the inhibition was at the level of transcription. The induction of COX2 in these cells presented an approach for evaluating polyamides as inhibitors of transcription. COX2 mRNA, COX2 protein, and PGE₂ levels all exist at very low levels prior to induction by IL-1β in synovial fibroblast cells, and would all remain at low levels after IL-1β induction in the presence of polyamides that prevent transcription of the COX2 gene.

Polyamides were designed to bind to DNA minor groove regions to disrupt binding of transcription factors that are known to bind to specific sequences in the human COX2 promoter. These include Ets-1, TATA box, LEF-1, NFkB and CRE binding sites. The examples below contain descriptions of these polyamides and their target binding sites. Ets-1, TATA box and LEF-1 sites were selected as initial targets for a combination of two polyamides to inhibit the binding of these three transcription factors to the HIV-1 promoter to reduce viral levels 99.9% in peripheral blood mononuclear cells compared to positive controls.

Several biological assays were available for evaluating COX2 transcription in these cells, including an ELISA assay of prostaglandin E₂ levels (PGE₂ synthesis requires COX2), Western analysis of COX2 protein levels, TaqMan and Northern analyses of COX2 mRNA levels, and an MTT assay of cell viability. MTT [3-(4,5-dimethlthiazol-2-yl) diphenyl tetrazolium bromide] is a pale yellow substrate that is cleaved by living cells to a dark blue formazan product by the mitochondrial enzyme succinate-dehydrogenase. The conversion takes place only in living cells and the amount of formazan produced is proportional to the number of cells present and the metabolic rate of the cell. Certain polyamides from these studies gave reductions in COX2 mRNA, COX2 protein, and PGE₂ levels. In all cases, inhibition was not due to any toxicity of the polyamide, since cell viability was found to be excellent after polyamide treatment. Certain other polyamides provided very large enhancement of COX2 mRNA, protein, and PGE₂ levels that were all very statistically significant. Collectively, these results indicate that polyamides can suppress or enhance COX2 mRNA levels in cells, and these changes correspond with similar changes in COX2 protein and PGE₂ levels. Mechanistically, these effects are consistent with a modulation of transcription of the COX2 gene.

Control experiments were conducted to determine the selectivity of these polyamides for the COX2 gene compared to IL-6 and ICAM1 (Intracellular Adhesion Molecule-1 or CD54), which are also induced by IL-1β in synovial fibroblasts. In the studies carried out, ICAM1 and IL-6 levels were unaffected by polyamides which suppressed COX2. Polyamides that enhanced COX2 levels did not affect ICAM1 levels, but did enhance IL-6 levels, although not to the same degree seen for PGE₂, COX2 mRNA, and COX2 protein. These results demonstrated that the polyamides studied were largely selective for COX2. Complete specificity for only the COX2 gene was not expected or achieved, though, because the polyamides in this work recognized the equivalent of only 5-7.5 base pairs, which corresponds to ˜3×10⁶ to 1×10⁵ perfect match binding sites, respectively, for these polyamides in the human genome. Not surprisingly, binding sites are present in the promoter regions of the ICAM1 and IL-6 genes. As expected, control polyamides, which did not target transcription factor binding sites in the COX2 promoter, did not suppress levels of PGE₂ and COX2 mRNA.

Surface Plasmon Resonance (BiaCore) binding data were also obtained for a set of polyamides targeted to the Ets-1 binding site. These studies showed very high binding of the polyamides to their intended target DNA sequence. No correlation between binding affinity and inhibition of COX2 was found.

In PK studies, polyamides were not orally available in rats but were present in blood plasma for up to 10 hours after intravenous dosing. These compounds were stable in mouse plasma at pH<1 for 10-12 hours at room temperature, which showed that the lack of oral bioavailability was not due to instability in acid. A follow up study with ¹⁴C-radiolabeled localization in and rate of clearance from rats.

Dosage

The aforementioned polyamide compounds may be administered in pharmaceutically acceptable concentrations to the cells or organisms possessing the target DNA according to methods known in the art. The more than one polyamide compound may be administered, separately, simultaneously, or sequentially to the cells or organisms. The route of administeration of the molecular trafficking compound may be administered orally, intravenously, intraperitoneally, subcutaneously, transdermally, and the like.

The dosing regimen of polyamide compounds in the present invention is selected in accordance with a variety of factors. These factors include the selected polyamide compound or compounds, the type, age, weight, sex, diet, and medical condition of the patient, the type and severity of the condition being treated with polyamide therapy, the target cell type being treated with polyamide therapy, the route of administration, pharmacological considerations such as the activity, efficacy, pharmacokinetics and toxicology profiles of the particular inhibitors employed, whether a drug delivery system is utilized, and whether the inhibitors are administered with other ingredients. Thus, the dosage regimen actually employed may vary widely and therefore deviate from the preferred dosage regimen set forth below.

Administration of the polyamide compounds may be with a regimen calling for a single daily dose, for multiple, spaced doses throughout the day, for a single dose every other day, for a single dose every several days, or other appropriate regimens.

The polyamides may be administered generally to an organism through oral or parenteral routes. The polyamide may also be administered by injection or catheter to localize the polyamides to specific organs or tissues containing the target cells to be treated by polyamide therapy. The polyamides may be prepared in physiologically acceptable media in an appropriate form for the route of administration. Polyamide compositions may be prepared as powders, solutions, and dispersions in media for both oral and parenteral routes of administration.

The polyamides should be administered at a dosage that provides a polyamide concentration of about 1 nM to about 1 mM in the intracellular or extracellular location of the target cells. Preferably the polyamides should be provided at a dosage that provides a polyamide concentration of about 1 nM to about 100 μM in the intracellular or extracellular location of the target cells, more preferably between about 10 nm to 10 μM. In order to attain a desired concentration of polyamides inside the cell, the concentration of polyamides outside the cell in the extracellular sera should be approximately 2 to 1000 times greater in concentration.

The polyamides may also be administered in combination with one or more additional therapeutic agents. Depending on the condition being treated, the combination therapy may also include antibiotics, vaccines, cytokines, other COX2 inhibitors, molecular trafficking compounds which facilitate cellular uptake and nuclear concentration of polyamides, and the like.

The following examples will further illustrate the invention.

### EXAMPLE 1

Polyamides Design and Synthesis for use In COX2 Transcription

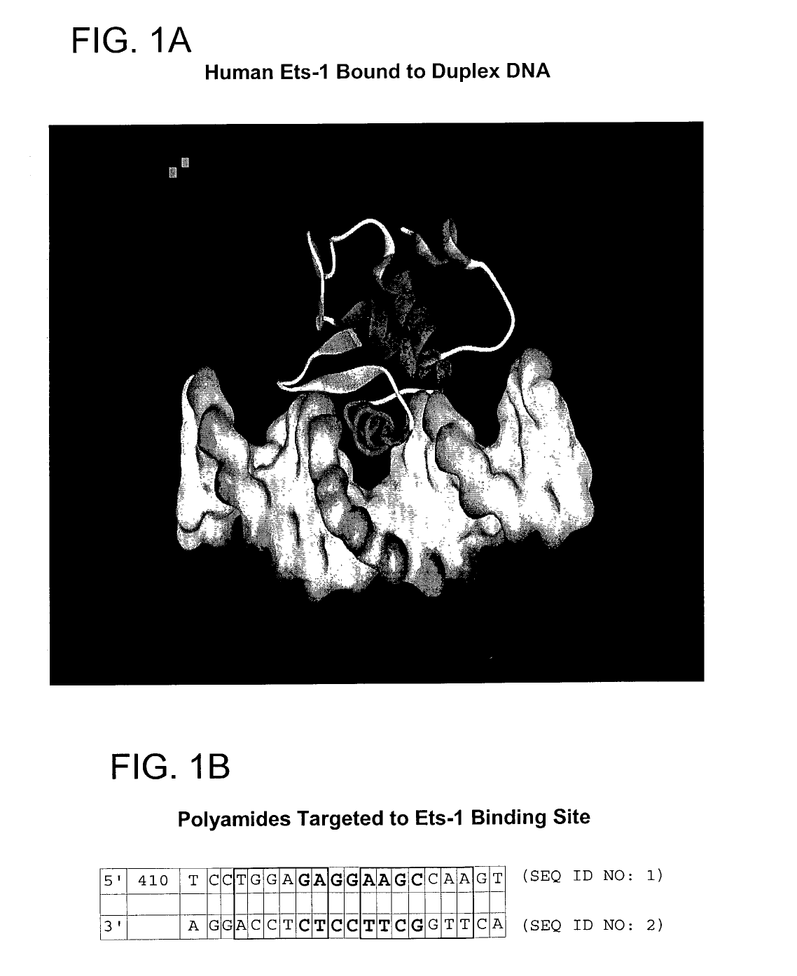

Polyamides were designed to bind to DNA minor groove regions that either partially or completely overlap DNA sequences where transcription factors bind to the COX2 promoter. Since transcription factor binding sites for a specific gene are flanked by unique DNA sequences, these flanking sequences were included in the polyamide targets to selectively inhibit the binding of the transcription factor to its COX2 binding site with minimal disruption of the transcription factor's binding to other promoters in the genome. For example, the ribbon structure in FIG. 1a shows human transcription factor Ets-1 bound to a segment of duplex DNA, via interaction of an a-helix of the protein with the major groove of the DNA. The actual sequence where Ets-1 binds in the human COX2 promoter is outlined in the sequence shown in FIG. 1b, and the sites where polyamides were designed to bind are in bold typeface. Using this approach, polyamides were also designed as inhibitors of the TATA box, NFkB, LEF-1 and CRE protein binding sites. Polyamide-DNA recognition was based on polyamide binding affinities to DNA, described above. All polyamides were targeted to 5′-(W)₁₋₂G(N)ₓW-3′ motifs, where X=3-6, W=A or T, and N=any nucleotide.

Polyamides targeted to the Ets-1, TATA box, and CRE sites suppressed PGE₂, COX2 protein, and COX2 mRNA levels. Polyamides targeted to the NFkB and LEF-1 sites were not inhibitors; in fact, some of these compounds actually enhanced PGE₂, COX2 protein, and COX2 mRNA levels.

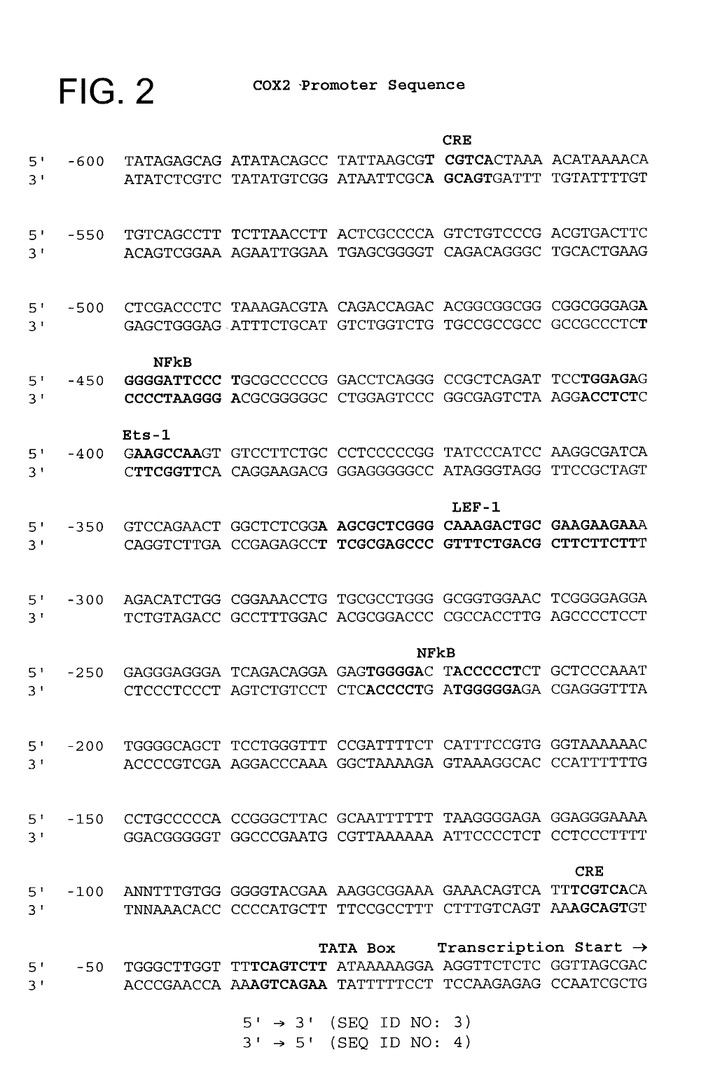

Polyamides were targeted to five transcription factor binding sites located in the first 600 bp of the human COX2 promoter as seen in FIG. 2. These transcription factor binding sites are labeled above the site in bold black type. Polyamides were synthesized to bind to the sequences that are in bold typeface.

Table 1 provides a listing of the polyamides that were synthesized for the COX2 promoter, and their DNA binding sites. These polyamides were prepared by solid phase synthesis and purified by reverse phase chromatography. They are grouped according to the transcription factor they were designed to inhibit. Abbreviations used in the table include W=A or T, Im=N-methylimidazole-2-carbonyl, -Im=4-amino-N-methylimidazole-2-carbonyl, -Py=4-amino-N-methylpyrrole-2-carbonyl, -γ=4-aminobutyryl, -β=3-aminopropionyl, -Dp=3-(dimethylamino)propylamino. Amide bonds (—CONH—) connect the polyamide subunits. The four polyamides with no transcription factor binding sites (No site) were used in control experiments.

TABLE 1

| Compound | Polyamide | DNA Binding Motif | TF Site |
|---|---|---|---|
| 1 | Im-Im-Py-Py-γ-Py-Im-Py-Py-β-Dp | 5′-WGGCTW-3′ | Ets-1 |
| 2 | Im-Im-β-Py-γ-Py-Im-Py-Py-β-Dp | 5′-WGGCTW-3′ | Ets-1 |
| 3 | Im-Im-Py-Py-γ-Py-Im-β-Py-β-Dp | 5′-WGGCTW-3′ | Ets-1 |
| 4 | Im-Im-Py-Im-γ-Py-Py-β-Py-β-Dp | 5′-WGGAGW-3′ | Ets-1 |
| 5 | Im-Im-β-Im-γ-Py-Py-Py-Py-β-Dp | 5′-WGGAGW-3′ | Ets-1 |
| 6 | Im-β-Py-Py-γ-Py-Im-Im-Py-β-Dp | 5′-WGCCAW-3′ | Ets-1 |
| 7 | Im-Py-Py-Py-Im-γ-Py-Py-Im-Py-Py-β-Dp | 5′-WGTCAGW-3′ | TATA |
| 8 | Im-Im-Im-Im-γ-Py-Py-β-Py-β-Dp | 5′-WGGGGW-3′ | NFkB |
| 9 | Im-Im-Im-β-Py-γ-Py-Py-Py-Py-Py-β-Dp | 5′-WGGGWWW-3′ | NFkB |
| 10 | Im-Im-Im-Py-Py-γ-Py-β-Py-Py-Py-β-Dp | 5′-WGGGWWW-3′ | NFkB |
| 11 | Im-Im-Im-Py-γ-Py-Py-Py-Py-β-Dp | 5′-WGGGWW-3′ | NFkB |
| 12 | Im-Im-Im-Py-γ-Py-β-Py-Py-β-Dp | 5′-WGGGWW-3′ | NFkB |
| 13 | Im-Im-Im-Py-γ-Py-Py-β-Py-β-Dp | 5′-WGGGWW-3′ | NFkB |
| 14 | Im-Im-Im-Im-Im-γ-Py-β-Py-Py-Py-β-Dp | 5′-WGGGGGW-3′ | NFkB |
| 15 | Im-β-Im-Py-γ-Im-β-Im-Py-β-Dp | 5′-WGCGCW-3′ | LEF-1 |
| 16 | Im-Py-Im-Py-γ-Im-Py-Im-Py-β-Dp | 5′-WGCGCW-3′ | LEF-1 |
| 17 | Im-Py-Py-Py-Im-γ-Py-Im-Im-Im-Py-β-Dp | 5′-WGCCCGW-3′ | LEF-1 |
| 18 | Im-β-Py-Py-Im-γ-Py-Im-Im-Im-Py-β-Dp | 5′-WGCCCGW-3′ | LEF-1 |
| 19 | Im-Py-Py-β-Im-Py-Im-γ-Py-Im-Py-β-Im-Py-Py-β-Dp | 5′-WGWWGCGW-3′ | LEF-1 |
| 20 | Im-Py-Py-γ-Im-Py-Py-β-Dp | 5′-WGWCW-3′ | LEF-1 |
| 21 | Im-Py-Py-Im-Py-γ-Py-Py-Py-Py-Py-β-Dp | 5′-WGWWGW-3′ | LEF-1 |
| 22 | Im-Py-Py-Im-γ-Py-Im-Py-Py-β-Dp | 5′-WGWCGW-3′ | CRE |
| 23 | Im-Py-Py-Im-γ-Py-Im-Im-Py-β-Dp | 5′-WGCCGW-3′ | No site |
| 24 | Im-β-Py-Im-γ-Py-Im-Im-Py-β-Dp | 5′-WGCCGW-3′ | No site |
| 25 | Im-β-Im-Py-γ-Py-Im-Py-Py-β-Dp | 5′-WGW?WW-3′ | No site |
| 26 | Im-Im-β-Py-γ-Im-Im-Py-Py-β-Dp | 5′-WGGCCW-3′ | No site |

In addition to experiments with individual polyamides, four mixtures were used in the experiments described in this report. Mixture 1=Compounds 1, 7, 17, and 19; Mixture 2=Compounds 1, 4, 7, and 22.

### EXAMPLE 2

Experimental Design for Obtaining Statistically Valid Data

Experiments with synovial fibroblast cells were carried out with mixtures of polyamides to maximize the chances of inhibiting COX2 through synergy of two or more compounds and to test polyamides in a small number of experiments. The two mixtures summarized in Table 2 each contained four polyamides targeted to a different set of transcription factor binding sites in the human COX2 promoter. Each mixture contained two polyamides that targeted the same transcription factor. Mixture 1, for example, contained one polyamide targeted to Ets-1, one polyamide targeted to TATA Box, and two polyamides targeted to LEF-1.

TABLE 2
Polyamide Binding Site Mixtures

| | Ets-1 | TATA Box | LEF-1 | CRE |
|---|---|---|---|---|
| Mixture 1 | Compound 1 | Compound 7 | Compound 17 | |
| | | | Compound 19 | |
| Mixture 2 | Compound 1 | Compound 7 | | Compound 2 |
| | Compound 4 | | | |

To obtain statistically valid data using these mixtures of polyamides, a randomized experimental design was used to measure suppression of COX2 mRNA & PGE₂ levels at 6 hours post (+)IL-1β stimulation, and COX2 protein & PGE₂ levels at 24 hours post (+)IL-1 stimulation of synovial fibroblast cells. The primary purpose of the randomized sample distribution was to avoid systematic errors in TaqMan, PGE₂ and Western analyses. Each randomized 12-well plate contained four wells of (+)IL-1 controls (no polyamides added), two wells of (−)IL-1 controls, three wells of one polyamide mixture, and three wells of another polyamide mixture. Cells were initially dosed with one of these mixtures at a total polyamide concentration of 20 μM (5 μM for each of the four polyamides in the mixture). After overnight incubation (˜16 hours), the media was removed and the cells were activated with IL-1β in media containing 20 μM of fresh polyamide mixture. These polyamide incubation times were chosen to optimize cellular uptake, based on in-house fluorescence microscopy work that showed polyamides did not enter undifferentiated U937 cells over a 2-3 hour period after polyamide treatment, but did enter these cells over a 24 hour period.

### EXAMPLE 3

Material and Methods for Cell Culture and Assay Conditions

Human rheumatoid synovial fibroblasts (RSFs) were maintained in DMEM (Gibco 11995-040 with pyridoxal HCl and glutamine, Life Technologies, Rockville, Md.), supplemented with 15% FBS, 1% glutamine, and 50 μg/ml gentamycin, with medium changes every 3 days, and incubated at 37° C. with 5% CO₂. Cells were passaged using trypsin containing 0.25% EDTA and propagated at 1:3 ratios; after passage number 25, a fresh culture was prepared from an aliquot of RSFs that was frozen at passage 12.

For assays, 12-well culture plates were inoculated with trypsinized cells at 40,000 cells per well in a volume of 2 ml. When wells were at near-confluency (˜120,000 cells/well after about 6 days), cells were allowed to preincubate overnight with the appropriate polyamide (PA) mixture (mixtures contain each PA component at 5 μM), except for control wells. Starting with this preincubation and thereafter, the regular media was replaced with low-FBS media (as above but with only 1% FBS). Wells were randomized to minimize edge effects that could cause systematic errors.

The next morning the media was replaced with fresh media containing fresh polyamide mixture, plus 1 ng/ml recombinant human IL-1β (cat. #201-LB, R&D Systems, Minneapolis, Minn.), except for the (±)IL-1β control wells which received fresh media (±)IL-1β but without polyamides. The plates were allowed to incubate for 24 hours, then the media was removed and kept at −80° C. for potential later use in cytokine or PGE₂ assay. The wells were washed immediately with 2 ml low-FBS media, then replaced with 0.5 ml low-FBS media enriched with arachidonic acid at 100 μM. This was well above the Kₘ for COX-2 and ensures that the PGE₂ produced will be proportional to the amount of COX-2 enzyme present, rather than rate-limited by insufficient substrate. After 1 hour, this media was removed as well, and either used immediately for PGE₂ release assay by EIA (see below) or frozen for later use as above.

Plates for PGE₂ assay were finished with a viability assay (see below). Identical plates were set up at the same time, if desired, for Western blotting, ICAM1 assay, or mRNA message level determination (see below).

### EXAMPLE 4

Material and Methods for Cell Viability Evaluation

Cell viability was evaluated using the MTT assay. MTT (3-(4,5-dimethlthiazol-2-yl)-) diphenyl tetrazolium bromide)(cat. # M-2128, Sigma Chemical Co., St. Louis, Mo.) is a pale yellow substrate that is cleaved by living cells to yield a dark blue formazan product by the mitochondrial enzyme succinate-dehydrogenase. The conversion takes place only in living cells and the amount of formazan produced is proportional to the number of cells present, and somewhat upon the metabolic rate of the cell, which is influenced by its treatment (IL-1β treated control RSFs consistently have slightly greater (˜10%) blue formazan deposition that the (−)IL-1β controls). Immediately after removal of media for the PGE₂ assay, wells were filled with 1 ml of 1 mg/ml MTT in low-FBS media, and returned to the incubator for 1 hour. This was aspirated, discarded, and replaced with 200 μL of isopropanol, which lysed the cells and dissolved the formazan crystals. Absorbance was measured on a ELISA plate reader with a test wavelength of 570 nm and a reference of 630 nm. Cell density was also used as an informed check on viability.

### EXAMPLE 5

Material and Methods For PGE₂ Enzyme Immuno-Assay (EIA)

The EIA for PGE₂ was based upon a protocol by Caymen Chemical Company (Ann Arbor, Mich.). Briefly, wells of a 96-well plate were coated overnight with donkey anti-mouse antibody (cat. #715-005-151, Jackson Immunoresearch, West Grove, Pa.). After washing, 50 μL of either sample (diluted if necessary in low-FBS media, above), or PGE₂ standards (typically 0.28 to 10 ng/ml, cat. #414014, Caymen Chem Co.) was added. This was followed by 50 μL of PGE₂-acetylcholinesterase tracer (Cat. #414010, Caymen Chemical Co.) and 50 μL of 150-fold diluted anti-PGE₂ monoclonal antibody (prepared in-house, stock 2B5, reference date Apr. 4, 1994). This was incubated overnight in a humidified container, then wells were washed and 200 μL of Ellman's reagent was added (cat. #400050, Caymen Chemical Co.). After 1-4 hours (dependent upon rate of color development), absorbance was measured on a ELISA plate reader at 405 nm. Standard curves were determined using a 4-parameter logistic fit.

### EXAMPLE 6

Materials and Methods for ICAM1 Assay by FACS

Intracellular adhesion molecule-1 (ICAM-1, also called CD54) is expressed on the surface of RSFs in response to IL-1β and can be quantified using facilitated cell sorting (FACS). At the end of treatment, cells in plate wells were trypsinized and transferred to 12×75 mm polystyrene tubes for FACS analysis. They were washed, aspirated, and to all but one of the tubes representing replicate wells for a given treatment, anti-CD54 domain 2 antibody, conjugated to phycoerythrin (PE) (murine IgG₁, Cat. #206-050, Ancell Corp., Bayport, Minn.) was added at 1 μL (˜0.5 μg) per tube in 350 μL buffer (PBS with 0.2% sodium azide and 2% FBS). To the remaining tubes was added isotype control (cat. #278-050, Ancell Corp.). Tubes were shaken for 30-60 minutes at 4° C. in the dark, then 2 ml of buffer was added, cells were pelleted at 300×g, aspirated, and resuspended into sheath buffer containing 0.5% methanol-free formaldehyde. After at least one hour, cells were analyzed by FACS with gating to screen for intact cells. Relative expression of ICAM1 was determined by comparing median fluorescence with corrections for isotype and (−)IL-1β controls.

### EXAMPLE 7

Materials and Methods for COX-2 Protein Expression Quantitation by Western Blotting

At the end of treatment, media was removed from a plate and 100 μL of 2× sample buffer was added per well (with 2% sodium dodecyl sulfate (SDS) and 10% b-mercaptoethanol, Cat. #ER33, Owl Separation Systems, Inc., Portsmouth N.H.), the mixture was swirled and contents of each well transferred to a 500 μL Eppendorf tube, and placed on a 100° C. heating block for 5 minutes. Sample 15 μL aliquots were subjected to SDS-polyacrylamide gel electrophoresis (SDS-PAGE) using 10-20% gradient gels (Invitogen (Novex), Carlsbad, Calif.). Proteins were transferred to nitrocellulose sheets by electroblotting as per the Novex protocol. Sheets were blocked for 1 hour using 5% milk in tris-buffered saline with 0.05% Tween 20 (TBS-Tween). The sheets were blotted with anti-COX-2 antibody (from rabbit, cat. #PG 27B, Oxford Biomedical Research, Oxford, Mich.) at a 1:2500 dilution in TBS-Tween containing 0.1% BSA overnight at 4° C. with rocking, then washed and blotted with a secondary horse-radish peroxidase (HRP) conjugated donkey anti-rabbit antibody (cat. #NA 934, Amersham Life Science, Arlington Heights, Ill.) at 1:5000 dilution for 30 minutes. After washing, protein bands were visualized using enhanced chemiluminescence with exposure to X-Omat AR film (Eastman Kodak Corp., Rochester, N.Y.). COX-2 protein relative to the (+)IL-1β control was quantified using a Model SI Densitometer with ImageQuant version 5.0 software (Molecular Dynamics, Inc., Sunnyvale, Calif.). Corrections for variations in lane loadings were made by reblotting for a background protein, actin, using a goat anti-actin antibody (cat. #sc1616, Santa Cruz Biotechnology, Inc., Santa Cruz, Calif.) at a 1:600 dilution, followed by a secondary HRP-conjugated swine anti-goat antibody (cat. #602-275, Boehringer Mannheim Corp., Indianapolis, Ind.) at 1:2,500 for 30 minutes. Actin was visualized and quantitated as above.

### EXAMPLE 8

mRNA Determination

To address the statistical robustness of our TaqMan assays, COX2 mRNA measurements were determined on (+)IL-1β stimulated synovial fibroblasts of low passage number and on LPS-stimulated U937 cells. Improved methods for isolating mRNA were also used. In these studies without polyamide treatment, COX2 and cyclophilin (control) mRNA levels were measured by TaqMan and compared in 12 replicates for (+)IL-1β stimulated synovial fibroblasts and LPS stimulated U937 cells. Very tight levels of cyclophilin and COX2 mRNA were measured for the 12 replicates of each cell type. This important experiment demonstrated that a minimum of 20-50% inhibition of transcription by polyamides could be measured with statistical confidence. TaqMan and Northern blot analyses were performed according to published protocols.

### EXAMPLE 9

Effects of Polyamides on Synovial Fibroblast Cells

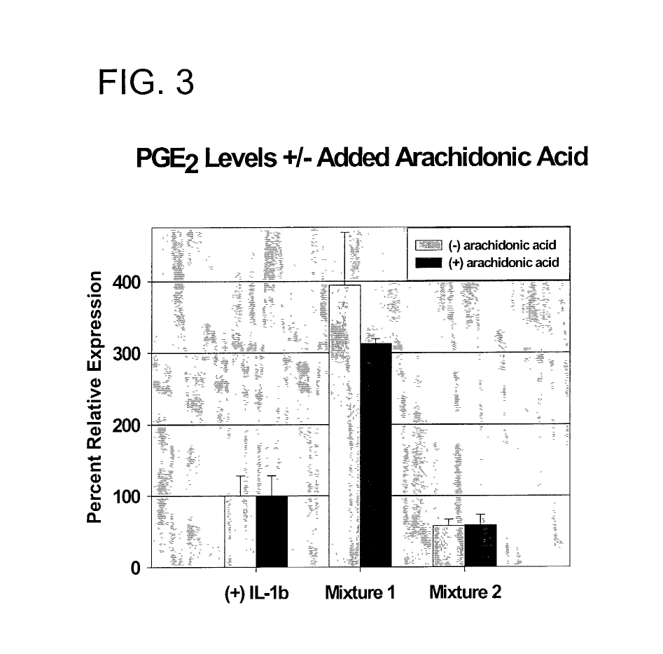

The effects of polyamide mixtures 1-2 on PGE₂, COX2 mRNA, COX2 protein, ICAM1 protein, and IL-6 protein levels were measured in synovial fibroblast cells. The results are summarized in this section and in FIGS. 3-8.

PGE₂ levels in the presence and absence of added arachidonic acid plus polyamide mixtures 1 or 2 were measured to determine whether any observed suppression of PGE₂ was due to decreased levels of the COX2 substrate, arachdonic acid (FIG. 3). In this experiment, to probe mechanism, IL-1β induced cells treated with polyamides and high levels of arachidonic acid were expected to suppress PGE₂ levels to the same extent as IL-1β induced cells treated with just polyamide, relative to controls. In the experiment, PGE₂ levels were determined 24 hours after (+)IL-1β stimulation, then the cell media was replaced with fresh media containing near saturating levels of arachidonic acid. PGE₂ levels in the media were again determined 1 hour later. Analysis clearly showed that arachidonic acid had no effect on PGE₂ levels compared to the (polyamide) untreated controls. Mixture 2 significantly suppressed PGE₂ levels: 55% without added arachidonic acid and 56% with added arachidonic acid. Surprisingly, Mixture 1 provided a very large enhancement in PGE₂ levels relative to its untreated control: 260% without added arachidonic acid and 330% with added arachidonic acid. All replicates in this statistically valid experiment showed the same enhancement with Mixture 1.

### EXAMPLE 10

Deconvolution of Polyamide Mixtures

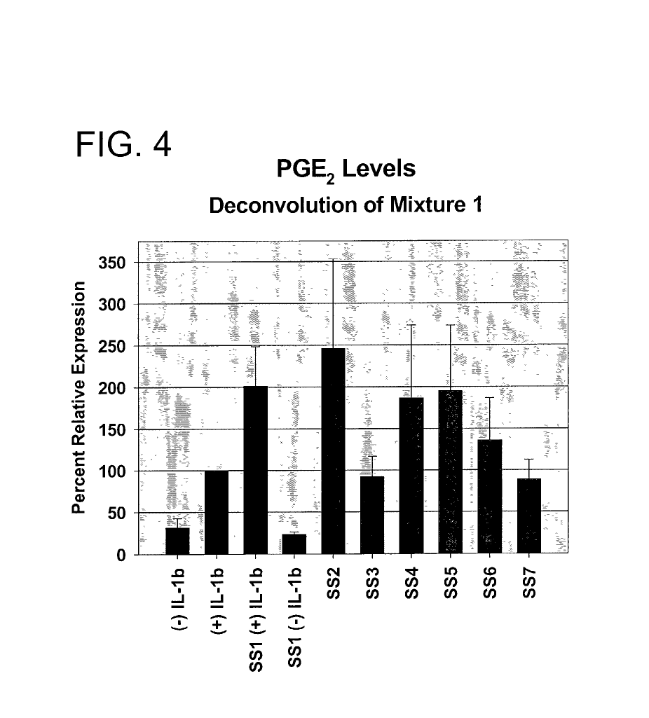

Deconvolution of Mixture 1 into subsets SS1-SS7 was conducted to determine which polyamide(s) was responsible for the increased inducement of the COX2 gene and enhanced PGE₂ levels. Mixture 1 contained one polyamide targeted to the Ets-l transcription factor, one polyamide targeted to the TATA box binding protein, and two polyamides targeted to different regions of a proposed LEF-1 binding site. In these deconvolution experiments, PGE₂ levels were measured for cells treated with all combinations of three polyamides. As shown in FIG. 4, only a single polyamide was in common among the subsets that enhanced PGE₂ levels (SS1, SS2, SS4, SS5, and SS6). This polyamide targeted the LEF-1 site and was not present in either mixture SS3 or SS7, neither of which enhanced PGE₂ levels. In addition, SS1 (which is Mixture 1) enhanced PGE₂ levels only when the cells were induced with IL-1β. These results indicate that polyamides are able to enhance gene transcription.

### EXAMPLE 11

Evidence of Polyamide-Regulated COX2 Transcription

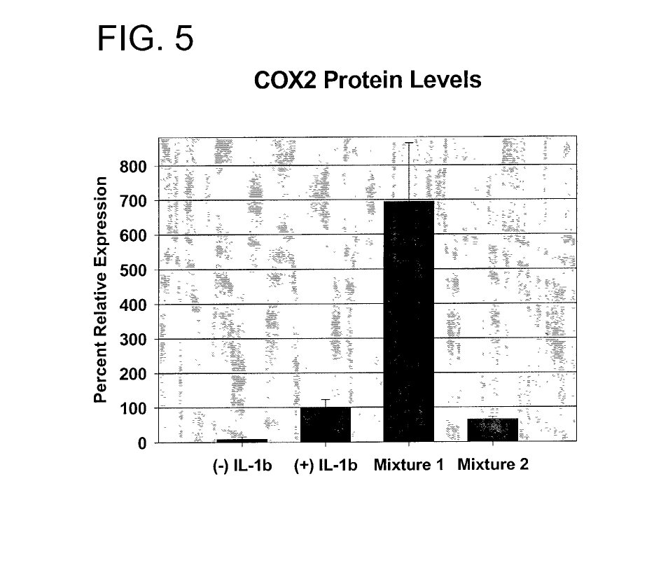

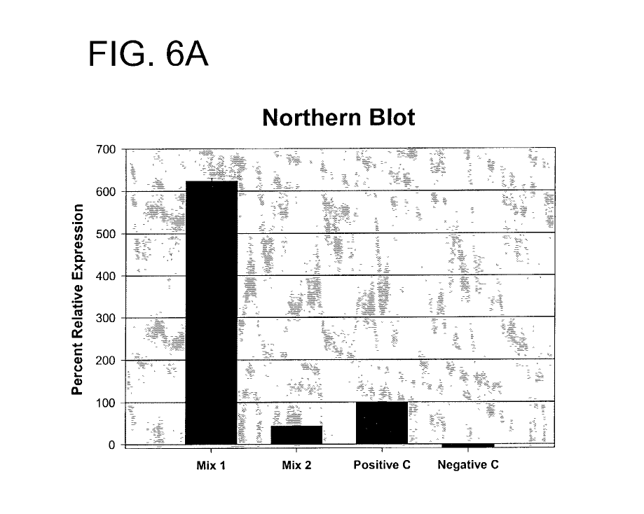

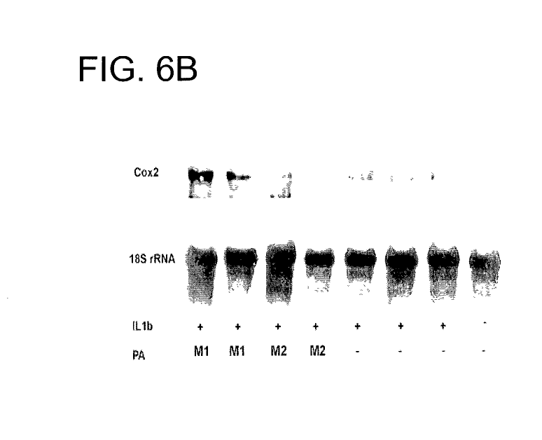

COX2 protein levels and COX2 mRNA levels in the presence of Mixtures 1 and 2 tracked with the PGE₂ levels described above. COX2 protein levels were assayed by Western analysis (FIG. 5) and COX2 mRNA levels were assayed by Northern blot (FIGS. 6a and 6b). TaqMan was not used to evaluate mRNA levels in these experiments. Like the PGE₂ levels, COX2 protein and COX2 mRNA levels were also significantly enhanced by Mixture 1. Compared to the untreated control, a 690% increase in COX2 protein levels was obtained with Mixture 1. A Northern blot confirmed that the enhancement of PGE₂ and COX2 protein levels was due to enhancement of transcription; more than a 6-fold increase in COX2 mRNA levels relative to 18S mRNA was found in treatments with Mixture 1. In contrast to these results, Mixture 2 provided 35% suppression of COX2 protein levels and 57% suppression of COX2 mRNA levels. These results were also in agreement with the corresponding PGE₂ suppression data. Significantly, the PGE₂, COX2 mRNA, and COX2 protein data obtained in three separate experiments clearly showed that polyamide-mediated changes in PGE₂ and COX2 protein levels correlated with COX2 mRNA levels. These results were consistent with polyamide regulation of transcription of the COX2 gene.

### EXAMPLE 12

Evidence of Polyamide Selectivity

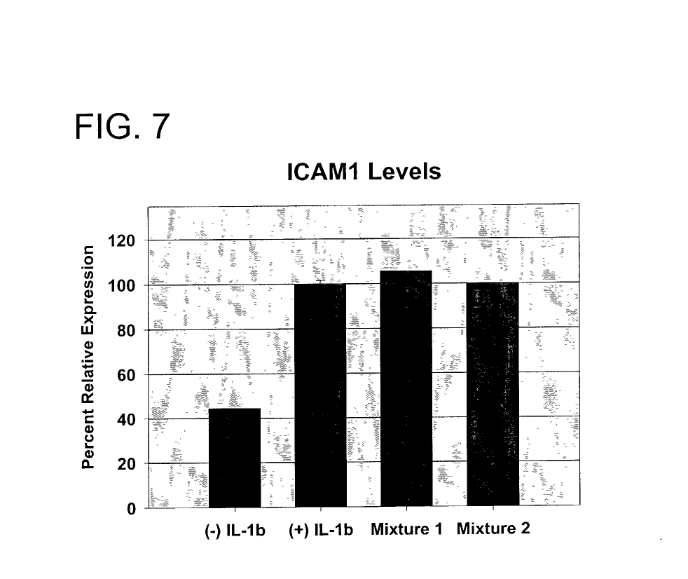

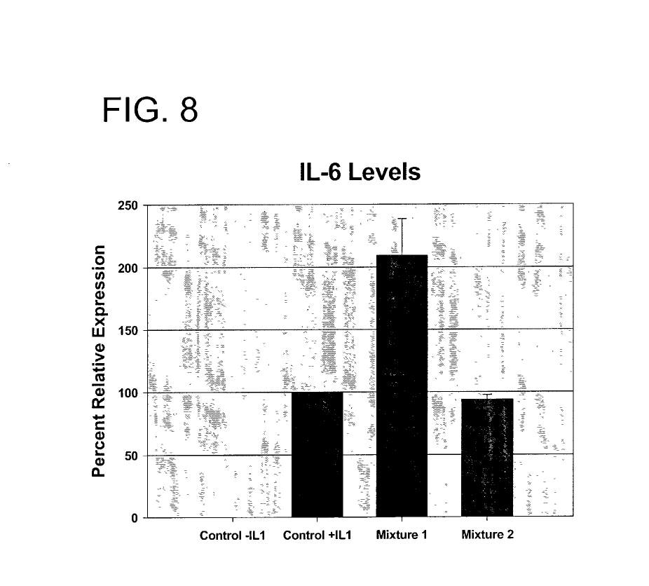

Selectivity for the COX2 gene versus ICAM1 and IL-6 genes was determined since these proteins are also induced in synovial fibroblasts by IL-1β. Complete gene specificity was not expected for these polyamides since their DNA recognition capabilities were on the order of 5-8 base pairs. ICAM1 levels were unaffected by mixtures 1 and 2 (FIG. 7). IL-6 levels were also unaffected by Mixture 2, but were enhanced by Mixture 1—though not to the same degree seen for PGE₂, COX2 mRNA, and COX2 protein (FIG. 8). These results demonstrated that the polyamides studied were selective for COX2. Other control polyamides not targeted to any transcription factor sites in the COX2 promoter had no inhibitory effects on COX2 mRNA levels in synovial fibroblast cells at 10 μM concentration, but did cause some inhibition at the lower concentration of 1 μM by TaqMan analysis. The same control polyamides did not suppress PGE₂ levels at either concentration, as measured by ELISA. These results indicated that transcription of the COX2 gene could be modulated selectively by polyamides targeted to transcription factor binding sites in the COX2 promoter.

### EXAMPLE 13

Biochemical Evidence of Polyamide Binding to DNA

Direct evidence that polyamides selectively bind to the targeted DNA sequences was obtained, as was proof that polyamides do not bind significantly to non-targeted DNA sequences. A 5′-biotinylated hairpin DNA sequence containing 6 bp of DNA flanking each side of the Ets-1 binding site was attached to a streptavidin chip, and BIAcore kinetic and thermodynamic values were obtained for a set of polyamides targeted to regions of this sequence. The kinetic on-rate constant (kₐ) and off-rate constant (k_{d}) and thermodynamic equilibrium constant (K_{Eq}) were determined from the association, dissociation and steady-state BIAcore measurements. The ratio kₐ/k_{d} was used to calculate an association constant K_{A} which was typically within a factor of 2 of the K_{Eq}, determined under steady state conditions. Values ranged from 2.7×10⁶ to 3.9×10⁸ M⁻¹. Calculated K_{D} values were as low as 0.8 nM, and were comparable to published dissociation constants of high affinity polyamides. A comparison of the BIAcore data with biological data showed no clear correlation between DNA binding constants and suppression of PGE₂ or mRNA levels. These results confirm that biological activity is due to a complex interplay of factors.

One potentially important factor is the kinetic dissociation constant (k_{d}), which is valuable for calculating the dissociation half-life of a polyamide from its duplex DNA complex. This constant was readily obtained by BIAcore measurements and provides a measure of the time it takes for a polyamide to dissociate from DNA. An effective inhibitor of transcription might need to have a long residence time on the specific operator sequence of DNA that it is designed to bind. If the polyamide rapidly dissociates and then re-binds to DNA, a transcription complex could form and initiate during the period when the polyamide is dissociated from the DNA. Under the dynamic conditions where polyamide-free buffer flowed past the chip surface where the DNA-polyamide complex was bound, the k_{d} ranged from 0.0049 to 0.16 sec⁻¹ for the Ets-1 targeted polyamides. Based on these k_{d} values, the calculated dissociation constants ranged from 4 seconds to 2.3 minutes.

### EXAMPLE 14

Pharmacokinetic Studies

Since polyamides are hoped to be suitable for use in animals, initial pharmacokinetic properties were obtained on a set of polyamides targeted to the Ets-1 and TATA box transcription factor binding sites in the human COX2 promoter. Each of 4 polyamides was evaluated orally in 3 rats at 5 mg/kg, and intravenous in 3 rats at 1 mg/kg. Blood was collected at timepoints ranging from 5 minutes to 24 hours post-application, and analyzed by for the presence of parent compound by LC-MS. In orally-dosed rats, polyamides were not detected in the plasma at any of the timepoints. In follow-up stability studies, these polyamides were found to be completely stable to mouse plasma at pH<1 for 10-12 hours at room temperature. In intravenous-dosed rats, the polyamides were cleared from the plasma over 10 hours.

In a related experiment, the concentrations of polyamides that remained in synovial fibroblast growth media used for determining PGE₂ levels were measured by LC-MS using the standard calibration curves generated from the rat PK studies. Two samples contained approximately ⅔ of their original polyamide concentration, a third contained approximately 1/10 of the original polyamide concentration, and a fourth contained none of the original polyamide. There was no correlation of these results with the rate of clearance of these compounds from plasma or the activity of these compounds as inhibitors of COX2 transcription.

In view of the above, it will be seen that the several objects of the invention are achieved.

As various changes could be made in the above compositions and processes without departing from the scope of the invention, it is intended that all matter contained in the above description be interpreted as illustrative and not in a limiting sense.

What is claimed is:

1. A process for regulating COX2 gene expression in a cell containing the COX2 gene the process comprising introducing a polyamide into the cell, the polyamide 5 comprising N-methyl pyrrole (Py) and N-methyl imidazole (IM) to provide specific binding to DNA at a COX2 gene promoter target site in a cell, wherein said polyamide binds to the COX2 gene promoter target site and regulates transcription of the COX2 gene.

2. The process of claim 1, wherein the polyamide binds sequences which partially or completely overlaps a transcription factor binding site for the COX2 gene.

3. The process of claim 2, wherein the transcription factor binding site is selected from the group consisting of CCRE, NFkB, Ets-1, LEF-1, and TATA box.

4. The process of claim 3 wherein the polyamide enhances the transcription of the COX2 gene.

5. The process of claim 4, wherein the transcription factor binding sites are selected from the group consisting of NFkB and LEF-1.

6. The process of claim 3, wherein the polyamide suppresses the transcription of the COX2 gene.

7. The process of claim 6, wherein the transcription factor binding sites are selected from the group consisting of CCRE, Ets-1, and TATA box.

8. The process of claim 3, wherein at least two different polyamides bind to one or more transcription factor binding site.

9. The process of claim 1, wherein the polyamide binds to the DNA singly or in pairs.

10. The process of claim 1, wherein the polyamide forms a hairpin turn.

11. A polyamide compound for regulating COX2 gene expression comprising N-methyl pyrrole (Py) and N-methyl imidazole (IM), said polyamide compound specifically binding to a COX2 gene promoter region of DNA.

12. The polyamide compound of claim 11 wherein the polyamides bind sequences which partially or completely overlaps a transcription factor binding site for the COX2 gene.

13. The polyamide compound of claim 12, wherein the polyamide compound binds to the transcription factor binding site is selected from the group consisting of CCRE, NFkB, Ets-1, LEF-1, and TATA box.

14. The polyamide compound of claim 13, wherein the transcription factor binding site is CCRE and the polyamide compound is Im-Py-Py-Im-γ-Py-Im-Py-Py-β-Dp.

15. The polyamide compound of claim 13, wherein the transcription factor binding site is Ets-1 and the polyamide compound is selected from the group consisting of Im-Im-Py-Py-γ-Py-Im-Py-Py-β-Dp, Im-Im-β-Py-γ-Py-Im-Py-Py-β-Dp, Im-Im-Py-Py-γ-Py-Im-β-Py-β-Dp, Im-Im-Py-Im-γ-Py-Py-β-Py-β-Dp, Im-Im-β-Im-γ-Py-Py-Py-Py-β-Dp, and Im-β-Py-Py-γ-Py-Im-Im-Py-β-Dp.

16. The polyamide compound of claim 13, wherein the transcription factor binding site is TATA box and the polyamide compound is Im-Py-Py-Py-Im-γ-Py-Py-Im-Py-Py-β-Dp.

17. The polyamide compound of claim 13, wherein the transcription factor binding site is NFkB and the polyamide compound is selected from the group consisting of Im-Im-Im-Im-γ-Py-Py-β-Py-β-Dp, Im-Im-Im-β-Py-γ-Py-Py-Py-Py-Py-β-Dp, Im-Im-Im-Py-Py-γ-Py-β-Py-Py-Py-β-Dp, Im-Im-Im-Py-γ-Py-Py-Py-Py-β-Dp, Im-Im-Im-Py-γ-Py-β-Py-Py-β-Dp, Im-Im-Im-Py-γ-Py-Py-β-Py-β-Dp, and Im-Im-Im-Im-Im-γ-Py-β-Py-Py-Py-β-Dp.

18. The polyamide compound of claim 13, wherein the transcription factor binding site is LEF-1 and the polyamide compound is selected from the group consisting of Im-β-Im-Py-γ-Im-β-Im-Py-β-Dp, Im-Py-Im-Py-γ-Im-Py-Im-Py-β-Dp, Im-Py-Py-Py-Im-γ-Py-Im-Im-Im-Py-β-Dp, Im-β-Py-Py-Im-γ-Py-Im-Im-Im-Py-β-Dp, Im-Py-Py-β-Im-Py-Im-γ-Py-Im-Py-β-Im-Py-Py-β-Dp, Im-Py-Py-γ-Im-Py-Py-β-Dp, and Im-Py-Py-Im-Py-γ-Py-Py-Py-Py-Py-β-Dp.
